(19) United States
(12) Patent Application Publication
(10) Pub. No.: US 2025/0093320 A1
(43) Pub. Date: Mar. 20, 2025

(54) METHOD FOR ESTIMATING ABUNDANCE AND DISTRIBUTION FEATURES OF ANTIBIOTIC RESISTANCE GENES IN SURFICIAL SEDIMENTS OF LAKE AND RESERVOIR

(71) Applicant: CHANGJIANG RIVER SCIENTIFIC RESEARCH INSTITUTE, Wuhan, Hubei (CN)

(72) Inventors: Li LIN, Wuhan, Hubei (CN); Xiong PAN, Wuhan, Hubei (CN); Lei DONG, Wuhan, Hubei (CN); Huan LI, Wuhan, Hubei (CN)

(73) Assignee: CHANGJIANG RIVER SCIENTIFIC RESEARCH INSTITUTE, Wuhan, Hubei (CN)

(21) Appl. No.: 18/007,471

(22) PCT Filed: Dec. 28, 2022
(86) PCT No.: PCT/CN2022/142991

§ 371 Date: Jan. 30, 2023

(30) Foreign Application Priority Data
Sep. 9, 2022 (CN) 202211105406.7

(51) Int. Cl. G01N 33/18; C12Q 1/689; G06Q 50/02

(52) U.S. Cl. CPC G01N 33/1826; C12Q 1/689; G01N 33/188; C12Q 2600/156; G06Q 50/02

(57) ABSTRACT

The present application provides a method for estimating abundance and distribution features of antibiotic resistance genes (ARGs) in surfacial sediments of lake and reservoir, including step 1 of obtaining an annual input total of nitrogen and phosphorus pollutants of each tributary flowing into the lake and reservoir; step 2 of constructing a linear regression equation between the abundance of each type of ARGs and the nitrogen and phosphorus discharge; and step 3 of calculating annual input total of nitrogen and phosphorus pollutants to be estimated for each geographical location of the lake and reservoir using inverse distance weighting interpolation analysis based on the annual input total of the nitrogen and phosphorus pollutants obtained in step 1, and substituting the calculated annual input total into the linear regression equation to estimate the abundance of each type of ARGs and analyze a distribution feature of the ARGs.

### CROSS-REFERENCE TO RELATED APPLICATION

This application claims priority to Chinese Patent Application No. 202211105406.7, filed on Sep. 9, 2022, the entire content of which is hereby incorporated by reference.

### TECHNICAL FIELD

The present application relates to the field of ecological environmental evaluation of rivers and lakes, and more particularly, to a method for estimating abundance and distribution features of antibiotic resistance genes in surficial sediments of lake and reservoir.

### BACKGROUND

Antibiotics eventually circulate in the water environment through sewage and medical waste, resulting in an increase in the selective pressure of bacteria in the water ecological environment, and inducing the formation of antibiotic resistance bacteria (i.e, ARB) and antibiotic resistance genes (i.e, ARGs) that are transferred to the pathogen of humans and animals, and become a new contaminant of more concern. Because the water flow is relatively slow, the water exchange period is long. The lake and reservoir are often rich in antibiotic pollution and are also rich in ARGs. Therefore, it is urgently necessary to investigate and master a pollution status of ARGs in the lake and reservoir, develop an appropriate ARGs abundance estimation method, and formulate a scientific prevention and control strategy.

The eutrophication of lake and reservoir provides a rich nutritional basis for the growth and reproduction of antibiotic-resistant bacteria and the enrichment of ARGs. The results showed that there was significant correlation between ARGs and nitrogen and phosphorus enrichment in water and sediment in fresh water environment. In terms of the lake and reservoir, most of the nutrients enriched in the water body and sediment are derived from the point source and non-point source pollution discharge at the periphery of the lake and reservoir, while the ARGs in the water body and sediment is partly and directly derived from the basin environment of the lake reservoir, and is transferred to the lake and reservoir along with the point source and non-point source pollution discharge at the periphery. Therefore, the abundance and distribution features of ARGs in the water environment of the lake and reservoir theoretically have a good correlation with the pollution discharge in the basin, and can be predicted by constructing a regression model between the abundance and distribution features and the pollution discharge.

### SUMMARY

In view of this, an embodiment of the present application provides a method for estimating abundance and distribution features of antibiotic resistance genes in surficial sediments of lake and reservoir, including:
a) obtaining an annual input total of nitrogen and phosphorus pollutants of each tributary flowing into the lake and reservoir;
b) obtaining abundance of each type of ARGs in the 0˜10 cm sediment of a surface layer of the lake and reservoir in the next year, and analyzing correlation between the abundance of ARGs and the annual input total of the surrounding nitrogen and phosphorus pollutants by using a geographical weighted regression model, so as to construct a linear regression equation between the abundance of each type of ARGs and the nitrogen and phosphorus discharge; and
c) calculating an annual input total of nitrogen and phosphorus pollutants to be estimated for each geographical location of the lake and reservoir using Inverse Distance Weighting (IDW) interpolation analysis based on the annual input total of the nitrogen and phosphorus pollutants obtained in step a), and substituting the calculated annual input total of the nitrogen and phosphorus pollutants to be estimated into the linear regression equation constructed in step b) to estimate the abundance of each type of ARGs corresponding to the geographical location of the lake and reservoir and analyze a distribution feature of the ARGs of the lake and reservoir.

### BRIEF DESCRIPTION OF THE DRAWINGS

FIG. 1 is a flow chart illustrating a method for estimating abundance and distribution features of antibiotic resistance genes in surficial sediments of lake and reservoir according to an embodiment of the present application.

### EMBODIMENTS OF THE PRESENT DISCLOSURE

In order that an object, a technical solution, and an advantage of the present application may be made clearer, an embodiment of the present application will be further described with reference to the accompanying drawings.

As used herein, the term “coupled” may refer, for example, to “connected.”

The eutrophication of lake and reservoir provides a rich nutritional basis for the growth and reproduction of antibiotic-resistant bacteria and the enrichment of ARGs. The results showed that there was significant correlation between ARGs and nitrogen and phosphorus enrichment in water and sediment in fresh water environment. In terms of the lake and reservoir, most of the nutrients enriched in the water body and sediment are derived from the point source and non-point source pollution discharge at the periphery of the lake and reservoir, while the ARGs in the water body and sediment is partly and directly derived from the basin environment of the lake reservoir, and is transferred to the lake and reservoir along with the point source and non-point source pollution discharge at the periphery. Therefore, the abundance and distribution features of ARGs in the water environment of the lake and reservoir theoretically have a good correlation with the pollution discharge in the basin, and can be predicted by constructing a regression model between the abundance and distribution features and the pollution discharge.

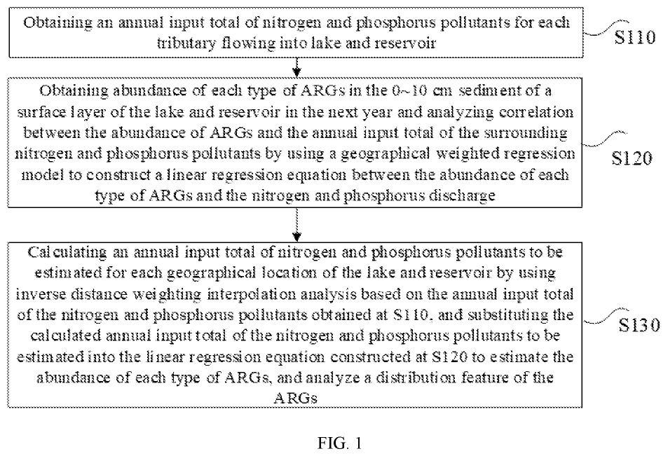

Referring to FIG. 1, a method for estimating abundance and distribution features of antibiotic resistance genes in surficial sediments of lake and reservoir according to an embodiment of the present application may include steps of S110-S130.

At S110, an annual input total of nitrogen and phosphorus pollutants for each tributary flowing into lake and reservoir is obtained. Th step may be implemented, for example, by a direct measurement method or a model estimation method as described below.

### \textcircled{1} Direct Measurement

Water bodies are monthly collected at all tributary inlets around the lake and reservoir to measure conventional water quality parameters while monitoring corresponding flow quantity and flow rate. An annual input total of nitrogen and phosphorus pollutants from each tributary is calculated by a product of the flow quantity and a concentration of the total nitrogen and the total phosphorus in the water quality parameters.

### \textcircled{2} Model Estimation

A pollution status of the lake and reservoir basin was analyzed by means of statistical yearbook, actual investigation, and the like, so as to establish a database of planting, rural living, scattered poultry breeding, urban living pollution, and the like. Using an output coefficient method, the annual input total of nitrogen and phosphorus pollutants polluted by such as poultry, a rural and urban life, and aquaculture is estimated by the following equation 1:

$$L = {\sum }_{i = 1}^{n} {E}_{i} [ {A}_{i} ( {I}_{i} ) ] + {p}_{1} \qquad ( 1 )$$

$${p}_{1} = cRQ$$

where L is the amount of nutrient; Eᵢ is an output coefficient of the i-th nutrient source; Aᵢ is the area of the i-th class land use type or the number of the i-th class livestock or population; Iᵢ is the nutrient input from the i-th nutrient source, p₁ is the nutrient input from the rainfall, and c is a nutrient concentration (g/m³) of the rainfall itself; R is annual rainfall (m³) in the basin; and Q is a rainfall runoff coefficient.

Further, an annual load total of total nitrogen (TN) and total phosphorus (TP) flowed into the lake and reservoir estimated by the output coefficient method for the poultry, the rural and urban life, and the aquaculture, etc., is coupled to a Soil and Water Assessment Tool (SWAT) hydrological model. Agricultural planting patterns in basin of the lake and reservoir including agricultural management measures such as planting, farming, irrigation, fertilization, etc., are introduced by applying a farmland management component of the SWAT hydrological model. A surface source pollution model of the basin of the lake and reservoir is established by loading the agricultural management measures and a point source pollution. The model is calibrated and validated using the measured water quality data on the basis of the parameter sensitivity analysis. Using the calibrated and validated model, the annual input total of the non-point source pollutant is simulated in a unit of month to obtain the monthly output total of total nitrogen and total phosphorus of each hydrologic research unit (i.e, HRU), and the annual output total of total nitrogen and total phosphorus per unit area of the sub-basin is obtained through watershed summarization and area conversion to calculate and analyze an annual input feature of nitrogen and phosphorus pollutants flowed into the tributary of the lake and reservoir.

At S120, abundance of each type of ARGs in the 0˜10 cm sediment of a surface layer of the lake and reservoir in the next year is obtained, and correlation between the abundance of ARGs and the annual input total of the surrounding nitrogen and phosphorus pollutants is analyzed by using a geographical weighted regression (GWR) model to construct a linear regression equation between the abundance of each type of ARGs and the nitrogen and phosphorus discharge.

For example, the total DNA of the sediment microorganisms can be extracted by collecting 0˜10 cm sediment of the surface layer of the lake and reservoir by a random uniform dot distribution method. Each type of ARGs gene-specific primer was used in combination with fluorescent quantitation Polymerase Chain Reaction (PCR) to determine the abundance of each type of ARGs in the surfacial sediment of each sampling point. According to the annual input total of the nitrogen and phosphorus pollutants at the peripheral tributary inlet of the sampling point obtained in step S110, the correlation between the abundance of ARGs and the peripheral nitrogen and phosphorus pollutants is analyzed by using a geographical weighted regression model, so as to constructing a geospatial relationship between the abundance of ARGs and the tributary pollution input. GWR can always perform regression analysis by the following equation 2 starting from the Ordinary Least Square (OLS) regression:

$${Y}_{i} = {β}_{0} ( {u}_{i} , {v}_{i} ) + {\sum }_{k = 1}^{{p}_{2} - 1} {β}_{k} ( {u}_{i} , {v}_{i} ) {X}_{ik} + {ε}_{i} , i = 1 , 2 , … , n \qquad ( 2 )$$

where, Yᵢ s a response variable, (uᵢ,vᵢ) represents coordinates of a spatial location i, β₀(uᵢ,vᵢ) and βₖ(uᵢ, vᵢ) represent an intercept and (p₂−1) slope parameters at the location i, respectively, Xᵢₖ represents (p₂−1) prediction variables at the location i, p₂ is a total number of parameters to be estimated, and εᵢ is an error term at the location i.

A linear regression equation between the abundance of each type of ARGs and the nitrogen and phosphorus discharge can be constructed according to an analysis result of the geographical weighted regression, as shown in equation 3 below, and the linear regression equation can be inversely verified using field survey data.

$${y}_{ARGs} = {ax}_{TN} - {bx}_{TP} + c \qquad ( 3 )$$

where, y_{ARGs} is the abundance of the antibiotic resistance gene ARGs at a point to be measured, X_{TN} is an annual total nitrogen input pollution load, x_{TP} is an annual total phosphorus input pollution load, and a, b, and c are intercepts of the linear regression equation.

At S130, an annual input total of nitrogen and phosphorus pollutants to be estimated for each geographical location of the lake and reservoir is calculated by using Inverse Distance Weighting (IDW) interpolation analysis based on the annual input total of the nitrogen and phosphorus pollutants obtained at S110, and the calculated annual input total of the nitrogen and phosphorus pollutants to be estimated is substituted into the linear regression equation constructed at S120 to estimate the abundance of each type of ARGs corresponding to the geographical location of the lake and reservoir, and analyze a distribution feature of the ARGs of the lake and reservoir.

In the process of specifically estimating the abundance and distribution features of the ARGs, the annual input total of the nitrogen and phosphorus pollutants for each tributary of the lake and reservoir in the previous year can be calculated according to the method in S120. On the basis of this, an inverse distance weighted interpolation analysis is performed according to the following equation 4 to calculate the annual input total of nitrogen and phosphorus pollutants virtually flowed into the lake and reservoir for each geographical coordinate point in the whole lake and reservoir water area of the lake and reservoir:

$$\hat{Z} ( {s}_{0} ) = {\sum }_{i = 1}^{N} {λ}_{i} Z ( {s}_{i} ) \qquad ( 4 )$$

$${λ}_{i} = \frac{{d}_{i 0}^{- P}}{{\sum }_{i = 1}^{N} {d}_{i 0}^{- P}}$$

$${\sum }_{i = 1}^{N} {λ}_{i} = 1$$

where \hat{Z}(s₀) represents an interpolation result at s₀, Z(sᵢ) is an annual pollution load value obtained at sᵢ, N is the number of tributaries of the lake and reservoir around which the interpolation is performed, λᵢ is a weight of each lake and reservoir tributary inlet used in a interpolation calculation process, dᵢ₀ is a distance between the interpolation point and each known lake and reservoir tributary inlet sᵢ, P is a weighted power index, and the sum of a weight λᵢ of each lake and reservoir tributary to an interpolation result is 1.

Further, the abundance of each type of ARGs at each geographical coordinate point of the lake and reservoir is estimated by substituting the annual input total of each geographical coordinate point of the lake and reservoir into the constructed linear regression equation to analyze the distribution feature of ARGs of the lake and reservoir.

In the case where the annual input total of pollutant for the entire lake and reservoir calculated in S110 is dispersed to a lake region, a particular point in the lake region has the total of pollutant received for one year at the point. The sum of the total of pollutant inputted at all points in the lake region is the total in S110. The abundance of the resistance gene at a particular point can be estimated based on a linear regression equation according to an annual input total of the pollutant at that point. In the case where the abundance of the resistance genes of all points in the lake region is known, the distribution features of the resistance genes in the lake region can be obtained.

The pollution concentration and distribution features of the ARGs in the surface sediments of the lake and reservoir can be easily, accurately and quickly estimated by constructing the geographical weighted regression model between the annual discharge total of nitrogen and phosphorus pollutants in each tributary of the basin and the measured abundance of the ARGs based on better correlation between the abundance and distribution features of the ARGs in the water environment of the lake and reservoir and the pollution discharge in the basin, thereby providing a scientific guidance for the pollution control and abatement of the ARGs in the lake and reservoir.

### EXAMPLE

Taking Erhai Lake as an example, the abundance and distribution features of ARGs in the surfacial sediments of Erhai Lake are estimated below.

The geographical and ecological environment of Erhai Lake is summarized as follows.

Erhai Lake (where north latitude is 25° 36′−25° 58′ and east longitude is 100° 06′-100° 18′) is the second largest plateau freshwater lake in Yunnan Province, located in a north-south intermountain basin, with an elevation of 1974 meters. The area of the water region is 252 square kilometers and an average water depth is 10.8 meters. The north is Eryuan basin and Dengchuan basin, and main rivers flowing into the lake include Miju River, Luoshi River and Yongan River. The west is the Tibetan-Yunnan fold system, the Diancang Mountain screen is located on the west side of the Erhai Lake, and main rivers flowing into the lake are Cangshan Shibaxi revers. The southeast rivers flowing into the lake are such as Balo River, Yulong River, Baita River, Fengtaiqing and so on. It is not only the main source of drinking water in Dali City, but also an important source of water for domestic use and industrial and agricultural production. It is an important force for regulating the climate of Dali City and promoting the agricultural development of the whole basin and even the sustainable development of the whole economy and society, and is called the “mother lake” of the Dali people. In recent years, the problem of water environment in Erhai Lake has become more and more serious. With the rapid development of agricultural industry in the basin, especially the rapid growth of economic crop planting area and poultry breeding scale, the amount of chemical fertilizer is increased, the structure of fertilization is unreasonable, etc., and poultry feces and urine have not been effectively utilized and treated, which threatens the water quality of Erhai Lake. Among them, nitrogen and phosphorus are the primary pollutants in Erhai Lake, and non-point source pollution in rural regions and farmland is an important cause of eutrophication in Erhai Lake. Hundreds of thousands of pigs, cattle, and sheep and millions of poultry are produced every year in the basin.

### 1) Determination of Pollution Load in Erhai Lake Basin in 2018

A pollution status of the Erhai lake basin was analyzed by means of statistical yearbook, actual investigation, and the like, so as to establish a database of planting, rural living, scattered poultry breeding, urban living pollution and the like. The survey scope covered 17 towns in Dali City and Eryuan County. The data came from Dali City Statistical Yearbook, Eryuan County Statistical Yearbook, Environmental Protection Statistical Yearbook, China Natural Resources Database and investigation data of various towns. Survey data is started in 2014 as the base year. The output coefficient method was used to respectively estimate the load amount of pollutants such as total nitrogen (TN) and total phosphorus (TP) in the poultry, the rural and urban life, and the aquaculture, etc.

A critical catchment area threshold is set to 5 km² based on the SWAT hydrological model in the Erhai Lake Basin to generate 545 sub-basins. The land use, soil type area threshold is set to 10% and the slope is 20%, to generate 1977 hydrological response units. The model of surface source pollution in Erhai Lake basin is established by loading agricultural management measures and point source pollution (poultry breeding and living pollution). The agricultural production mode and fertilization mode of 500 households in Erhai Lake basin are statistically recorded. According to the statistical result, a representative mainstream agricultural production mode is selected in each town of Erhai Lake basin for scenario simulation from fourrotation modes including broad bean-rice, garlic-rice, garlic-corn and rape-rice. 2014 will be used as a model warm-up period and 2015-2016 will used as calibration and validation periods. A lake entrance of Miju River is selected as a water quality verification station, and the water quantity, total nitrogen and total phosphorus data of the lake entrance of Miju River are used in a model calibration phase from January to December 2015, and the corresponding data of the lake entrance of Miju River are used in a model verification phase from January to December 2016. On the basis of the verification of hydrological parameter calibration, the concentrations of total nitrogen and total phosphorus in the river are calibrated.

According to the characteristics of the sewage interception project around the lake, the pollution of rural life and poultry breeding is subtracted from the surface source pollution model of the Erhai Lake basin, and six sewage disposal plants (point source pollution) are added. the calibrated and verified model is used to simulate surface source pollution load in 2018 in a unit of month to obtain a month output amount of the total nitrogen and total phosphorus of each of the hydrological response unit, and the annual average output amount of total nitrogen and total phosphorus per unit area of the sub-basin is obtained by basin summarization and area conversion. In 2018, the rainfall in Erhai Lake basin was 1000.7 mm, and the precipitation was concentrated from July to October. Because the catchment area of the rivers flowing into the lake is relatively smaller, the fluctuation law of runoff and rainfall is almost the same. The simulation results of TN and TP of each river flowing into the Erhai Lake in 2018 are shown in Table 1.

TABLE 1
Annual input amount of TN and TP of each
river flowing into ErHai Lake in 2018

| | | | Coordinates of entrance | |
|---|---|---|---|---|
| | TN | TP | flowing into the Lake | |
| Tributary name | (tom ) | (ton ) | East Longitude | North latitude |
| Yangnang River | 2.136 | 0.233 | 100.2246881 | 25.62233509 |
| Tingming River | 0.336 | 0.011 | 100.2170062 | 25.63848925 |
| Mocan River | 31.597 | 0.529 | 100.2107084 | 25.67140022 |
| Qinghi River | 10.699 | 0.093 | 100.2106118 | 25.67525842 |
| Heilong River | 20.818 | 0.253 | 100.2079082 | 25.68320165 |
| Bahe River | 19.717 | 1.117 | 100.2053118 | 25.69629743 |
| Zhonghe River | 9.298 | 0.933 | 100.191493 | 25.71725539 |
| Tao River | 3.548 | 0.077 | 100.1800346 | 25.73094181 |
| Mei River | 0.599 | 0.023 | 100.1763278 | 25.7336818 |
| Yinxian River | 1.049 | 0.061 | 100.172857 | 25.73452746 |
| Shuangyuan | 0.813 | 0.058 | 100.1637697 | 25.73909878 |
| River | | | | |
| Baishi River | 0.776 | 0.137 | 100.1534271 | 25.74988369 |
| Lingquan River | 1.463 | 0.058 | 100.1491141 | 25.7602811 |
| Jin River | 4.464 | 0.153 | 100.1495218 | 25.76729595 |
| Mangyong River | 6.907 | 0.270 | 100.1466894 | 25.79598872 |
| Yang River | 2.489 | 0.201 | 100.1490927 | 25.81569288 |
| Wanhua River | 8.020 | 0.424 | 100.1356387 | 25.86078758 |
| Xiayi River | 0.518 | 0.036 | 100.1178074 | 25.89787402 |
| Zongshu River | 0.566 | 0.037 | 100.1156616 | 25.90864446 |
| Yulong River | 9.654 | 0.786 | 100.2583122 | 25.70712488 |
| Fengtaoqing | 24.401 | 0.428 | 100.2195168 | 25.82858573 |
| River | | | | |
| Boluo River | 190.109 | 25.824 | 100.2718735 | 25.61676284 |
| Banta River | 12.710 | 1.351 | 100.2808106 | 25.63827887 |
| Yongan River | 11.826 | 0.809 | 100.1578903 | 25.95248818 |
| Luoshi River | 92.086 | 6.011 | 100.1008987 | 25.94230061 |
| Miju River | 591.624 | 36.335 | 100.1342011 | 25.9287158 |

2) Geographical Weighted Regression Relationship Analysis Between Nitrogen and Phosphorus Pollution Load Flowing into Erhai Lake and Abundance of Antibiotic Resistance Gene (ARGs) in Surfacial Sediments of Erhai Lake
\textcircled{1} survey of ARGs distribution features of surfacial sediments In Erhai Lake

Ten sampling points were randomly and evenly disposed in Erhai, and the coordinates of each of the sampling points are shown in Table 2. In March 2019, 0˜10 cm sediment was collected from a surface layer of each point to extract the total DNA of the sediment microorganisms. Each type of ARGs gene-specific primer is used in combination with fluorescence quantitative PCR (qPCR) to determine the abundance of each type of ARGs in the surfacial sediment of each sampling point. The results are shown in Table 2 below.

TABLE 2
Actual investigation results of abundance of the ARGs (copies/16S
rRNA copy) in surfacial sediments in Erhai Lake in 2019

| | | | Total | ARGs gene class | | | | | | |
|---|---|---|---|---|---|---|---|---|---|---|
| Number | Longitude | Latitude | ARGs | Amin | β-Lactam | FCA | MGE | MLSB | Sulf | Tet |
| E1 | 100.1142883 | 25.93087712 | 0.03701 | 0.00054 | 0.00022 | 0.01735 | 0.00467 | 0.00059 | 0.01355 | 0.00009 |
| E2 | 100.147934 | 25.94384423 | 0.03650 | 0.00102 | 0.00030 | 0.01315 | 0.00306 | 0.00028 | 0.01859 | 0.00010 |
| E3 | 100.1434708 | 25.91790858 | 0.03609 | 0.00270 | 0.00098 | 0.00796 | 0.00595 | 0.00093 | 0.01707 | 0.00050 |
| E4 | 100.1527405 | 25.87127181 | 0.01759 | 0.00032 | 0.00039 | 0.01009 | 0.00309 | / | 0.00346 | 0.00024 |
| E5 | 100.2059555 | 25.87559652 | 0.01957 | 0.00040 | 0.00027 | 0.01113 | 0.00171 | / | 0.00554 | 0.00053 |
| E6 | 100.1881027 | 25.78814479 | 0.01454 | 0.00021 | 0.00002 | 0.00944 | 0.00344 | 0.00002 | 0.00119 | 0.00022 |
| E7 | 100.1881027 | 25.7341892 | 0.02171 | 0.00033 | 0.00027 | 0.01449 | 0.00196 | / | 0.00445 | 0.00021 |
| E8 | 100.223465 | 25.69970044 | 0.01666 | 0.00014 | 0.00008 | 0.01157 | 0.00116 | / | 0.00364 | 0.00006 |
| E9 | 100.2674103 | 25.6693792 | 0.01695 | 0.00034 | 0.00007 | 0.01058 | 0.00277 | / | 0.00310 | 0.00008 |
| E10 | 100.2399445 | 25.60809439 | 0.01846 | 0.00103 | 0.00034 | 0.00682 | 0.00198 | / | 0.00801 | 0.00028 |

Where Amin refers to an aminoglycoside; B-Lactam refers to B-lactam; MGE refers to a moveable gene element; Sulf refers to sulfonamides; and Tet refers to tetracycline.

\textcircled{2} Geographical weighted regression analysis

According to the annual load amount of nitrogen and phosphorus pollution load of 26 tributaries in the periphery of 10 sampling points in Erhai Lake, correlation between the abundance of ARGs in Table 2 and the nitrogen and phosphorus pollution load of 26 tributaries flowing into Erhai Lake in Table 1 is analyzed by using the geographical weighted regression model, so as to construct a geospatial relationship between the abundance of each type of ARGs in the lake and reservoir and pollution input from the tributaries. A linear regression equation between the abundance of each type of ARGs and nitrogen and phosphorus discharge is constructed according to the results of the geographical weighted regression analysis. The results are shown in Table 3.

TABLE 3
Linear regression analysis between abundance of ARGs in surfacial sediments of
Erhai Lake and nitrogen and phosphorus discharge from tributaries of Erhai Lake

| | | calibration | |
|---|---|---|---|
| ARGs type | Linear regression equation | R² | P |
| Total ARGs | y_{ARGs} = 0.000214x_{TN} − 0.001275x_{TP} + 0.013171 | 0.954 | 0.000191 |
| FCA | y_{FCA} = 0.000083x_{TN} − 0.000684x_{TP} + 0.009631 | 0.929 | 0.000585 |
| Sulf | y_{Sul} = 0.000091x_{TN} − 0.000381x_{TP} + 0.001408 | 0.968 | 0.000078 |
| Amin | Y_{Amin} = 0.000006x_{TN} − 0.000007x_{TP} + 0.000117 | 0.961 | 0.000131 |
| β-Lactam | y_{β-Lactam} = 0.000002x_{TN} − 0.000008x_{TP} + 0.000112 | 0.850 | 0.003761 |
| MGEs | y_{MGEs} = 0.000027x_{TN} − 0.000166x_{TP} + 0.001776 | 0.856 | 0.003370 |
| MLSB | y_{MLSB} = 0.000004x_{TN} − 0.000026x_{TP} − 0.000089 | 0.963 | 0.000115 |
| Tet | y_{Tet} = 0.000001x_{TN} − 0.000003x_{TP} − 0.000205 | 0.332 | 0.157260 |

Where, a unit of x is ton and a unit of y is copies/16S rRNA copy.

\textcircled{3} Abundance and Distribution of ARGs in Surface Sediments of Erhai Lake

In the process of specifically estimating the abundance and distribution features of the ARGs of Erhai Lake, the load total of the nitrogen and phosphorus pollutants of each tributary flowing into Erhai Lake in the previous year is calculated according to the method in step 1. On the basis of this, an inverse distance weighted interpolation analysis is used to calculate load amount of nitrogen and phosphorus pollutants virtually flowed into the lake and reservoir for each geographical coordinate point in the Erhai Lake, and the calculated TN and TP are substituted into the linear regression equation of the corresponding gene in Table 3, so as to calculate the abundance of ARGs at various locations in the Erhai Lake, thereby judging the distribution features of ARGs.

The examples of the present application have been described in detail above, but the content is only a preferred embodiment of the present application and should not be considered as limiting the scope of implementation of the present application. Any modifications or substitutions can be readily conceivable by those skilled in the art within the technical scope of the disclosure of this application, without departing from the spirit and scope of the technical solution of this application, which are intended to be encompassed within the scope of the claims of this application. Accordingly, the scope of protection of the present application is defined by the appended claims.

What is claimed is:

1. A method for estimating abundance and distribution features of antibiotic resistance genes (ARGs) in surfacial sediments of lake and reservoir, comprising:
a) obtaining an annual input total of nitrogen and phosphorus pollutants of each tributary flowing into the lake and reservoir;
b) obtaining abundance of each type of ARGs in the 0˜10 cm sediment of a surface layer of the lake and reservoir in the next year, and analyzing correlation between the abundance of ARGs and the annual input total of the surrounding nitrogen and phosphorus pollutants by using a geographical weighted regression model, so as to construct a linear regression equation between the abundance of each type of ARGs and the nitrogen and phosphorus discharge; and
c) calculating annual input total of nitrogen and phosphorus pollutants to be estimated for each geographical location of the lake and reservoir using inverse distance weighting interpolation analysis based on the annual input total of the nitrogen and phosphorus pollutants obtained in step a, and substituting the calculated annual input total of the nitrogen and phosphorus pollutants to be estimated into the linear regression equation constructed in step b to estimate the abundance of each type of ARGs corresponding to the geographical location of the lake and reservoir and analyze a distribution feature of the ARGs of the lake and reservoir.

2. The method of claim 1, wherein the step a includes:
estimating the annual input total of nitrogen and phosphorus pollutants of each tributary flowing into the lake and reservoir by using a pollutant annual input total estimation model based on a status of a social and economic production activity in a studied basin of the lake and reservoir; or
calculating the annual input total of nitrogen and phosphorus pollutants of each tributary flowing into the lake and reservoir by using water quality and hydrological monitoring data of the tributary flowing into the lake and reservoir.

3. The method of claim 2, further comprising: estimating, by the pollutant annual input total estimation model applying an output coefficient method, a total nitrogen (TN) and total phosphorus (TP) pollutant index load amount of poultry, rural and urban life, and aquaculture pollution, respectively, from a pollutant generation stage, a pollutant loss stage, and a pollutant inflow stage, and couple the estimated pollutant index load amount to a SWAT hydrological model to simulate the annual input total of the nitrogen and phosphorus pollutant of the tributary flowing into the lake and reservoir.

4. The method of claim 2, wherein when the pollutant annual input total estimation model coupled to a SWAT hydrological model applies a farmland management component of the SWAT hydrological model, the method further comprises: determining, by the farmland management component, farm production time, fertilization time, and fertilization amount to introduce agricultural planting patterns in the basin of the lake and reservoir including agricultural management measures and estimate farmland soil pollutant amount flowing into the lake and reservoir in combination with rainfall time and rainfall amount of the basin, wherein the agricultural management measures include planting, farming, irrigation, fertilization.

5. The method of claim 2, wherein the pollutant annual input total estimation model uses an output coefficient method to estimate the annual input total of nitrogen and phosphorus pollutants of each tributary flowing into the lake and reservoir by a following equation:
$$L = {\sum }_{i = 1}^{n} {E}_{i} [ {A}_{i} ( {I}_{i} ) ] + {p}_{1}$$
$${p}_{1} = cRQ$$
wherein, L is the amount of nutrient, which refers to the annual input total of the pollutants; Eᵢ is an output coefficient of the i-th nutrient source; Aᵢ is the area of the i-th class land use type or the number of the i-th class livestock or population; Iᵢ is the nutrient input from the i-th nutrient source, p₁ is the nutrient input from the rainfall, and c is a nutrient concentration (g/m³) of the rainfall itself; R is annual rainfall (m³) in the basin; and Q is a rainfall runoff coefficient.

6. The method of claim 1, wherein the correlation between the abundance of ARGs and the annual input total of the nitrogen and phosphorus pollutants in the peripheral tributaries is analyzed in the step b using the geographical weighted regression model to construct a geospatial relationship between the distribution feature of each type of ARGs in the lake and reservoir and the pollution input, i.e., a linear regression equation between the abundance of each type of ARGs and the nitrogen and phosphorus discharge, wherein the geographical weighted regression model always performs regression analysis by a following equation starting from the Ordinary Least Square regression:
$${Y}_{i} = {β}_{0} ( {u}_{i} , {v}_{i} ) + {\sum }_{k = 1}^{{p}_{2} - 1} {β}_{k} ( {u}_{i} , {v}_{i} ) {X}_{ik} + {ε}_{i} , i = 1 , 2 , … , n$$
wherein, Yᵢ is a response variable, (uᵢ,vᵢ) represents coordinates of a spatial location i, β₀(uᵢ,vᵢ) and βₖ(uᵢ,vᵢ) represent an intercept and (p₂−1) slope parameters at the location i, respectively, Xᵢₖ represents (p₂−) prediction variables at the location i, p₂ is a total number of parameters to be estimated, and εᵢ is an error term at the location i.

7. The method of claim 1, wherein, in the step b, the linear regression equation represented by a following equation is constructed using the abundance of each type of ARGs in the surfacial sediments of field-investigated lake and reservoir, and the constructed linear regression equation is inversely validated using the abundance of each type of ARGs in the surfacial sediments of field-investigated lake and reservoir:
$${y}_{ARGs} = {ax}_{TN} - {bx}_{TP} + c \qquad ( 3 )$$
wherein, y_{ARGs} is the abundance of the antibiotic resistance gene ARGs at a point to be measured, x_{TN} is an annual total nitrogen input pollution load, x_{TP} is an annual total phosphorus input pollution load, and a, b, and c are intercepts of the linear regression equation.

8. The method of claim 1, wherein the inverse distance weighting interpolation analysis is performed according to a following equation:
$$\hat{Z} ( {s}_{0} ) = {\sum }_{i = 1}^{N} {λ}_{i} Z ( {s}_{i} )$$
$${λ}_{i} = \frac{{d}_{i 0}^{- P}}{{\sum }_{i = 1}^{N} {d}_{i 0}^{- P}}$$
$${\sum }_{i = 1}^{N} {λ}_{i} = 1$$
wherein, \hat{Z}(s₀) represents an interpolation result at s₀, Z(sᵢ) is an annual pollution load value obtained at sᵢ, N is the number of tributaries of the lake and reservoir around which the interpolation is performed, λᵢ is a weight of each lake and reservoir tributary inlet used in a interpolation calculation process, dᵢ₀ is a distance between the interpolation point and each known lake and reservoir tributary inlet sᵢ, P is a weighted power index, and the sum of a weight λᵢ of each lake and reservoir tributary to an interpolation result is 1.
